(19) United States
(12) Patent Application Publication
(10) Pub. No.: US 2010/0233774 A1
(43) Pub. Date: Sep. 16, 2010

(54) Circulatory Biomass Energy Recovery System and Method

(76) Inventors: Tatsuki Toda, Tokyo (JP); Norio Nagao, Tokyo (JP); Norio Kurosawa, Tokyo (JP)

Correspondence Address: BIRCH STEWART KOLASCH & BIRCH, PO BOX 747, FALLS CHURCH, VA, 22040-0747, US

(21) Appl. No.: 12/225,387

(22) PCT Filed: Mar. 22, 2007
(86) PCT No.: PCT/JP2007/055876

§ 371 Date: Sep. 19, 2008

(30) Foreign Application Priority Data
Mar. 22, 2006 (JP) 2006-079341
Mar. 22, 2006 (JP) 2006-079342

(51) Int. Cl. C12P 5/02; F02C 3/28; C12M 1/107

(52) U.S. Cl. 435/167; 60/780; 435/292.1

(57) ABSTRACT

A circulatory biomass energy recovery system and method raising an energy recovery efficiency are provided. The circulatory biomass energy recovery system has a culture unit 10 filled with a culture solution culturing phytoplankton as a biomass material, a biomass material recovery unit 11 recovering the biomass material from the culture unit, an energy source conversion unit 12 converting the biomass material to an energy source capable of energy recovery, an energy recovery unit 13 recovering the energy from the energy source converted at the energy source conversion unit 12, and a carbon dioxide recovery unit 14 returning the carbon dioxide produced in the energy recovery unit 13 to the culture unit and is configured so that the energy source conversion unit 12 includes a methane fermentation unit 22 performing methane fermentation of the biomass material and a hydrogen production unit 21 by photosynthesis bacteria using the biomass material.

### TECHNICAL FIELD

The present invention relates to a circulatory biomass energy recovery system and method, more particularly relates to a circulatory biomass energy recovery system and method forming a closed circulation system using unicellular phyceae or other phytoplankton as a biomass material and recovering biomass energy.

### BACKGROUND ART

With global environmental problems now in the international spotlight, preservation of the natural environment, effective use of energy resources, and maintenance of the ecosystem have become important issues common to the human race. Regarding energy, energy using coal, petroleum, and other fossil fuels, energy utilizing natural wind power and solar power, nuclear power, and other energy sources may be mentioned as representative ones.

Here, other than wind power and solar power and other natural energy and nuclear energy, all energy is obtained by burning some material, therefore carbon dioxide is exhausted. However, carbon dioxide promotes global warming, therefore an energy source not discharging global warming gases including carbon dioxide has been desired.

On the other hand, with solar power generation by solar batteries, there is no exhaust of carbon dioxide at the time of electric power generation, but the large energy consumption in the process of production of the semiconductor silicon for the solar batteries is becoming a problem. Further, the nuclear power generation uses energy in the processes of mining, transportation, and disposal of the material.

In view of the above energy problems, in recent years, biomass energy has come into the spotlight.

“Biomass energy” indicates the organic matter stored as plants and other living thing (biomass) and regarded as an energy source. That is, plants perform photosynthesis, absorb carbon dioxide (CO₂), and convert it to biomass. When that biomass is burned, it is possible to recover heat energy.

The carbon dioxide exhausted at the time of burning of biomass is exhausted in only a fixed amount by the photosynthesis. Therefore, there is no exhaust of carbon dioxide in all processes from synthesis of the organic matter by photosynthesis to its combustion. For this reason, biomass energy is attracting attention as a clean energy.

Biomass energy is present in huge stocks on the earth and, at the same time, is constantly being produced by life performing photosynthesis by solar energy on the land and in the oceans.

By trial calculation, the stock of the biomass existing on the earth is 100 times the commercial energy consumed by the human race each year. The biomass flow produced year by year is 10 times that amount. In this way, biomass energy has properties of both sides of stock and flow and has the feature of being massive in amount.

The forms of use of biomass energy may be roughly classified into the “plantation type” of producing sugarcane, eucalyptus, corn, or other living thing (biomass) in a certain constant area and utilizing methane fermentation etc. to obtain energy and the “waste recovery type” of obtaining energy from the waste biomass such as garbage collected at waste disposal sites etc. and sludge.

Here, an energy recovery system using the above biomass energy will be explained.

FIG. 1 is a schematic view showing the processes of an energy recovery system using the biomass energy described above.

First, the biomass is produced and, if necessary, the biomass is harvested.

Biomass energy covers matter which contains carbon and can generate energy at the time of combustion, therefore there are a variety of such materials. Almost all organic matter falls under this category. As the plantation type, for example, use is made of wood harvested from pine/cedar forests etc., corn and sugarcane, eucalyptus, and other plants. As the waste recovery type, use is made of black solution generated in the process of production of wood pulp, bagasse produced after squeezing sugarcane, waste matter of domestic animals and other farm waste, and food waste and other city waste.

Next, the biomass produced as described above is converted to obtain methane gas, ethanol, oil, methanol, or other energy. This includes biochemical conversion and thermochemical conversion.

The solid waste produced as a result of the energy conversion is utilized at farmlands etc. while the wastewater is disposed of into the rivers and oceans.

The biomass energy described above is utilized in a manner of gathering biomass energy scattered over the earth at one location to recover the energy and is utilized in a matter of producing plants and other biomass at certain arable land, the sea, or another area and converting the same to energy in those locations.

The increasingly popular biomass energy in recent years has been almost all of the “waste recovery type” recovered from the sludge discharged from sewage treatment plants, garbage, and other waste. There is no practical example of the plantation type.

Waste type biomass is spreading because it is originally collected for disposal and the energy can be extracted before incineration, burial in landfills, or other disposal, so there is the advantage that the biomass can be recovered without excess recovery and transportation energy.

However, when envisioning the new energy for future measures against global warming for achieving the target values of the Kyoto Protocol, the energy recovered from only waste system biomass is small in absolute amount and just of an extent utilized for an energy-saving measures of waste disposal plants.

However, the conventional plantation type biomass energy recovery system had the following problems.

(1) Rather than selling electricity as energy, raising crops is more economical, so utilization as farmland is given a higher priority.

(2) The energy fixing efficiency per unit area is low and vast farmland is necessary. Further, when the biomass is directly burned for use, as shown by the graph of the moisture content of the biomass and the effective calorific value of FIG. 2, if the moisture content is high as in wet biomass etc., a high energy is required for evaporation, therefore the originally low energy density of the material becomes further lower and the effective calorific value ends up falling, so it is necessary to produce dry biomass having a low moisture content.

(3) The energy required for the harvesting and transportation of energy crops is large and the recovery efficiency of the biomass is low.

(4) Disposal of the residue from methane fermentation requires large energy. Wastewater and, in the process of power generation, carbon dioxide etc. are exhausted and applies a load on the environment.

Under the above circumstances, a circulatory biomass energy recovery system raising the energy recovery efficiency was developed. This was disclosed in Japanese Patent Publication (A) No. 2004-113087.

The above circulatory biomass energy recovery system is configured to culture phytoplankton as a biomass material in a culture unit filled with a culture solution, recover the biomass material cultured in the culture unit at a biomass material recovery unit, convert the biomass material to an energy source capable of recovering energy in an energy source conversion unit, and have the energy recovery unit recover the energy from the energy source converted at the energy source conversion unit. After this, it uses a carbon dioxide recovery unit to return the carbon dioxide generated in the energy recovery unit to the culture unit.

According to the circulatory biomass energy recovery system described in Japanese Patent Publication (A) No. 2004-113087, the following effects can be given:

(1) The desert and other unused land can be utilized, so there is no competition with agriculture.

(2) In comparison with land plants, by culturing a large amount of phytoplankton having a fast carbon fixing speed per unit area, a high energy yield can be obtained.

(3) The residue from methane fermentation is recycled in a culture tank of the phytoplankton as a fertilizer ingredient, therefore wastewater is not discharged outside of the system and the energy used for spreading the fertilizer can be reduced.

(4) The carbon dioxide discharged from electric power generation is reabsorbed in the culture tank of the phytoplankton, therefore carbon dioxide is not discharged outside of the system.

Further improvement of the energy recovery efficiency has been demanded from the energy recovery system utilizing biomass described above.

Further, the phenomenon where the phytoplankton suddenly dies in the culture tank as a whole due to a virus etc. during high density continuous culturing of the phytoplankton used as the biomass material is known.

If the phytoplankton suddenly dies, the speed of production of the phytoplankton used as the biomass material is lowered and stable supply becomes difficult. This may cause unstable supply of electric power or other energy.

### DISCLOSURE OF INVENTION

### Problem to be Solved by the Invention

The problem to be solved by the present invention is the difficulty of raising the energy recovery efficiency in a circulatory biomass energy recovery system and method.

Further, it is difficult to avoid destabilization of the supply of energy due to sudden death of the phytoplankton.

### Means for Solving the Problem

A circulatory biomass energy recovery system of the present invention has a culture unit filled with a culture solution for culturing phytoplankton as a biomass material, a biomass material recovery unit for recovering the biomass material from the culture unit, an energy source conversion unit for converting the biomass material to an energy source capable of energy recovery, an energy recovery unit for recovering energy from the energy source converted at the energy source conversion unit, and a carbon dioxide recovery unit for returning the carbon dioxide produced in the energy recovery unit to the culture unit, wherein the energy source conversion unit includes a methane fermentation unit for performing methane fermentation of the biomass material and a hydrogen production unit by photosynthesis bacteria using the biomass material.

The circulatory biomass energy recovery system of the present invention described above cultures the phytoplankton as the biomass material in the culture unit filled with the culture solution, recovers the biomass material from the culture unit in the biomass material recovery unit, converts the biomass material to an energy source capable of energy recovery in the energy source conversion unit, and recovers the energy from the energy source converted at the energy source conversion unit in the energy recovery unit. After this, the carbon dioxide recovery unit returns the carbon dioxide produced in the energy recovery unit to the culture unit.

Here, the energy source conversion unit includes a methane fermentation unit for performing methane fermentation of the biomass material and a hydrogen production unit by photosynthesis bacteria using the biomass material.

In the circulatory biomass energy recovery system of the present invention described above, preferably the energy source conversion unit includes a biomass solubilization unit for solubilizing the biomass material, a supernatant liquid of the biomass solution obtained by the biomass solubilization is supplied to the hydrogen production unit by the photosynthesis bacteria, and a precipitated part of the biomass solution is supplied to the methane fermentation unit.

In the circulatory biomass energy recovery system of the present invention described above, preferably the photosynthesis bacteria obtained in the hydrogen production unit by the photosynthesis bacteria and dead bodied thereof are supplied as the methane fermentation material to the methane fermentation unit.

In the circulatory biomass energy recovery system of the present invention described above, preferably hydrogen sulfide obtained in the methane fermentation unit is supplied to the hydrogen production unit by the photosynthesis bacteria and is utilized by the photosynthesis bacteria.

In the circulatory biomass energy recovery system of the present invention described above, preferably the energy recovery unit includes an electric power generation unit for generating electric power by burning the methane generated in the methane fermentation unit.

In the circulatory biomass energy recovery system of the present invention described above, preferably the energy recovery unit includes an electric power generation unit for generating electric power by burning the hydrogen produced in the hydrogen production unit by the photosynthesis bacteria.

In the circulatory biomass energy recovery system of the present invention described above, preferably the energy recovery unit includes a hydrogen recovery unit for recovering the hydrogen produced in the hydrogen production unit by the photosynthesis bacteria.

Further, a circulatory biomass energy recovery method of the present invention has a step of culturing phytoplankton as a biomass material in a culture unit filled with a culture solution, a step of recovering the biomass material from the culture unit, an energy source converting step of converting the biomass material to an energy source capable of energy recovery, an energy recovery step of recovering the energy from the energy source, and a step of recovering the carbon dioxide generated in the energy recovery step and returning it to the culture unit, wherein the energy source converting step includes a step of generating methane by methane fermentation of the biomass material and a step of producing hydrogen by photosynthesis bacteria using the biomass material.

The circulatory biomass energy recovery method of the present invention described above cultures phytoplankton as a biomass material in a culture unit filled with a culture solution, recovers the biomass material from the culture unit, converts the biomass material to an energy source capable of energy recovery, and recovers energy from the energy source. Further, it recovers carbon dioxide generated in the energy recovery step and returns it to the culture unit.

Here, the energy source converting step includes a step of generating methane by methane fermentation of the biomass material and a step of producing hydrogen by photosynthesis bacteria using the biomass material.

In the circulatory biomass energy recovery method of the present invention described above, preferably the energy source converting step includes a step of solubilizing the biomass material, a supernatant liquid of a biomass solution obtained by the solubilization of the biomass is used for hydrogen production by the photosynthesis bacteria, and a precipitated part of the biomass solution is used for the methane fermentation.

In the circulatory biomass energy recovery method of the present invention described above, preferably the photosynthesis bacteria obtained in the hydrogen production step by the photosynthesis bacteria and dead bodies thereof are used as the methane fermentation material.

In the circulatory biomass energy recovery method of the present invention described above, preferably the hydrogen sulfide obtained in the methane fermentation step is utilized by the photosynthesis bacteria in the hydrogen production step by the photosynthesis bacteria.

In the circulatory biomass energy recovery method of the present invention described above, preferably the energy recovery step includes a step of generating electric power by burning the methane produced by the methane fermentation.

In the circulatory biomass energy recovery method of the present invention described above, preferably the energy recovery step includes a step of generating electric power by burning the hydrogen produced in the hydrogen production by the photosynthesis bacteria.

In the circulatory biomass energy recovery method of the present invention described above, preferably the energy recovery step includes a step of recovering the hydrogen produced in the hydrogen production by the photosynthesis bacteria.

Further, to attain the above object, a circulatory biomass energy recovery system of the present invention has a plurality of culture units filled with a culture solution for culturing phytoplankton as a biomass material, a biomass material recovery unit for recovering the biomass material cultured in the plurality of culture units, an energy source conversion unit for converting the biomass material to an energy source capable of energy recovery, an energy recovery unit for recovering the energy from the energy source converted at the energy source conversion unit, and a carbon dioxide recovery unit for returning the carbon dioxide produced in the energy recovery unit to the culture units.

The circulatory biomass energy recovery system of the present invention described above cultures phytoplankton as a biomass material in a plurality of culture units filled with a culture solution, recovers the biomass material cultured in each of the plurality of culture units in a biomass material recovery unit, converts the biomass material to an energy source capable of energy recovery in the energy source conversion unit, and recovers the energy from the energy source converted at the energy source conversion unit in the energy recovery unit. After this, a carbon dioxide recovery unit returns carbon dioxide produced in the energy recovery unit to the culture units.

In the circulatory biomass energy recovery system of the present invention described above, preferably the plurality of culture units are constituted by partitioning one culture tank into a plurality of regions by partition members.

In the circulatory biomass energy recovery system of the present invention described above, preferably a monitor unit for monitoring a cultured state of the phytoplankton in each of the plurality of culture units is provided.

More preferably, the monitor unit includes a measurement unit for measuring a fluorescence intensity of an in vivo chlorophyll fluorescence by using the phytoplankton as a sample and finding a fluorescence quantum yield.

Still more preferably, when the cultured state of the phytoplankton becomes lower than a target level, the culture solution is replaced by a new one in any culture unit where the cultured state becomes lower than the target level among the plurality of culture units.

In the circulatory biomass energy recovery system of the present invention described above, preferably the culture units are continuous culture units for continuously culturing the phytoplankton.

Further, the circulatory biomass energy recovery method of the present invention has a step of culturing phytoplankton as a biomass material in a plurality of culture units filled with a culture solution, a step of recovering the biomass material cultured in the culture units, an energy source converting step of converting the biomass material to an energy source capable of energy recovery, an energy recovery step of recovering the energy from the energy source, and a step of recovering carbon dioxide generated in the energy recovery step and returning it to the culture units.

The circulatory biomass energy recovery method of the present invention cultures phytoplankton as a biomass material in a plurality of culture units filled with a culture solution, recovers the biomass material cultured in the plurality of culture units, converts the biomass material to an energy source capable of energy recovery, and recovers the energy from the energy source. After this, it recovers carbon dioxide generated in the energy recovery step and returns it to the culture units.

In the circulatory biomass energy recovery method of the present invention described above, preferably, as the plurality of culture units, use is made of a plurality of culture units constituted by partitioning one culture tank into a plurality of regions by partition members.

In the circulatory biomass energy recovery method of the present invention described above, preferably the step of culturing the phytoplankton is carried out while monitoring the cultured state of the phytoplankton in each of the plurality of culture units.

Further preferably, in the step of culturing the phytoplankton a fluorescence intensity of in vivo chlorophyll fluorescence is measured by using the phytoplankton as a sample and a fluorescence quantum yield is found.

Still more preferably, in the step of culturing the phytoplankton, when the cultured state of the phytoplankton becomes lower than a target level, the culture solution is replaced by a new one in any culture unit where the cultured state becomes lower than the target level among the plurality of culture units.

In the circulatory biomass energy recovery method of the present invention described above, preferably in the step of culturing the phytoplankton the phytoplankton is continuously cultured in the culture units.

### ADVANTAGEOUS EFFECTS

The circulatory biomass energy recovery system according to the present invention uses a methane fermentation unit performing methane fermentation of a biomass material and a hydrogen production unit by photosynthesis bacteria using the biomass material. The photosynthesis bacteria generate hydrogen and the photosynthesis bacteria themselves further become the biomass material of the methane fermentation and contribute to the increase of the recovered energy so enable the energy recovery efficiency to be raised.

Further, the circulatory biomass energy recovery method according to the present invention, when converting the energy source, performs methane fermentation of a biomass material and hydrogen production by photosynthesis bacteria using the biomass material. The photosynthesis bacteria generate hydrogen. Further, the photosynthesis bacteria themselves become the biomass material of the methane fermentation to contribute to the increase of the recovered energy and raise the energy recovery efficiency to enable recovery of the biomass energy.

Further, according to the circulatory biomass energy recovery system according to the present invention, by having a plurality of culture units, even when sudden death of the phytoplankton occurs in any culture unit among those, the influence exerted upon the other culture units can be avoided, a stable supply of the phytoplankton as the biomass material becomes possible, and stabilization of the electric power or other energy supply can be realized.

Further, according to the circulatory biomass energy recovery method according to the present invention, by culturing the phytoplankton in a plurality of culture units, even when sudden death of the phytoplankton occurs in any culture unit among those, the influence exerted upon the other culture units can be avoided, stable supply of the phytoplankton as the biomass material becomes possible, and stabilization of the electric power or other energy supply can be realized.

### BRIEF DESCRIPTION OF DRAWINGS

FIG. 1 is a schematic view showing processes of an energy recovery system using biomass energy according to a conventional example.

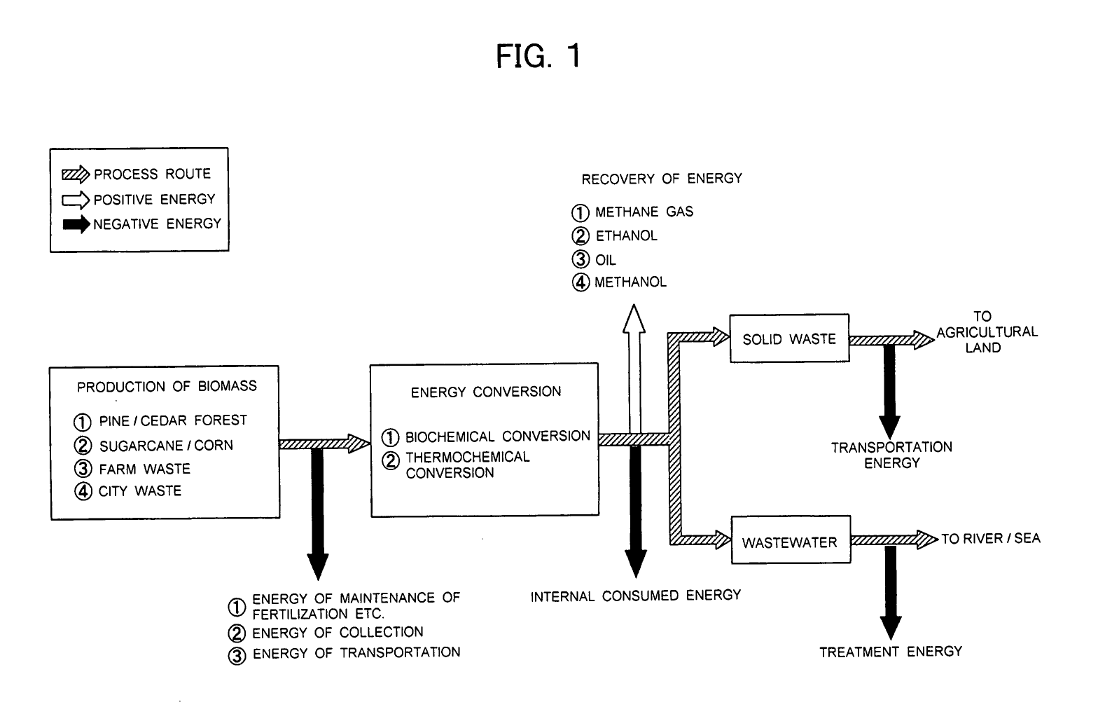

FIG. 2 is a diagram showing a relationship between a moisture content of the biomass and an effective calorific value.

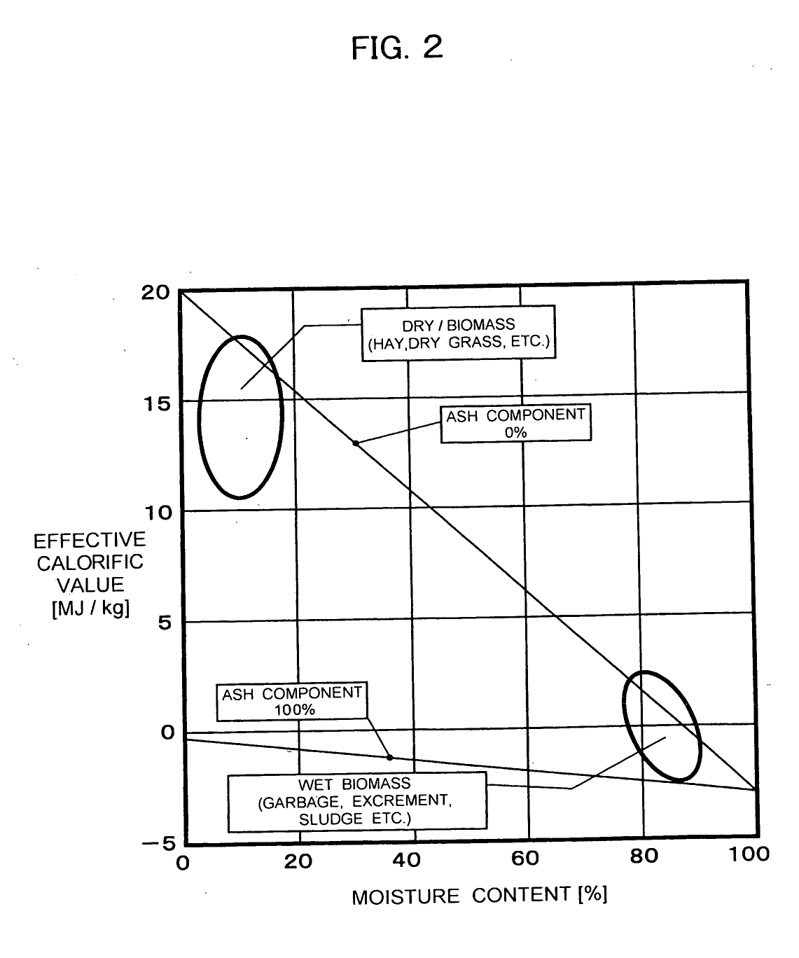

FIG. 3 is a view of the schematic constitution of a circulatory biomass energy recovery system according to a first embodiment of the present invention.

FIG. 4 is a view of the schematic constitution showing the constitution of an energy source conversion unit in FIG. 3 in the first embodiment of the present invention in detail.

FIG. 5 is a view of the schematic constitution showing the constitution of a culture unit according to a second embodiment of the present invention in detail.

FIG. 6 is a view of the schematic constitution showing a circulatory biomass energy recovery system given a more concrete constitution in the second embodiment of the present invention.

### EXPLANATION OF REFERENCE NUMERALS

10 . . . culture unit, 11 . . . biomass material recovery unit, 12 . . . energy source conversion unit, 13 . . . energy recovery unit, 14 . . . carbon dioxide recovery unit, 15 . . . nutrient recovery and conversion unit, 10a . . . continuous culture unit of phytoplankton, 11a . . . biomass material condensing and recovering unit, 12a . . . methane fermentation unit, 13a . . . electric power generation unit, 14a . . . carbon dioxide recovery unit, 15a . . . nutrient recovery and conversion unit, 16a . . . ammonia recovery unit, 20 . . . biomass solubilization unit, 21 . . . hydrogen production unit of photosynthesis bacteria, 22 . . . methane fermentation unit, 23 . . . ammonia recovery unit, 30 . . . methane fermentation and electric power generation unit, 100a to 100d . . . culture units, 101 . . . monitor unit, 102 . . . drainage system, 103 . . . new water supply source, and 104 . . . wild strain culture tank.

### BEST MODE FOR CARRYING OUT THE INVENTION

Below, embodiments of the circulatory biomass energy recovery system and method of the present invention will be explained with reference to the drawings.

### First Embodiment

The present invention is a plantation type circulatory biomass energy recovery system and a circulatory biomass energy recovery method using the same. Embodiments of the present invention will be explained below with reference to the drawings.

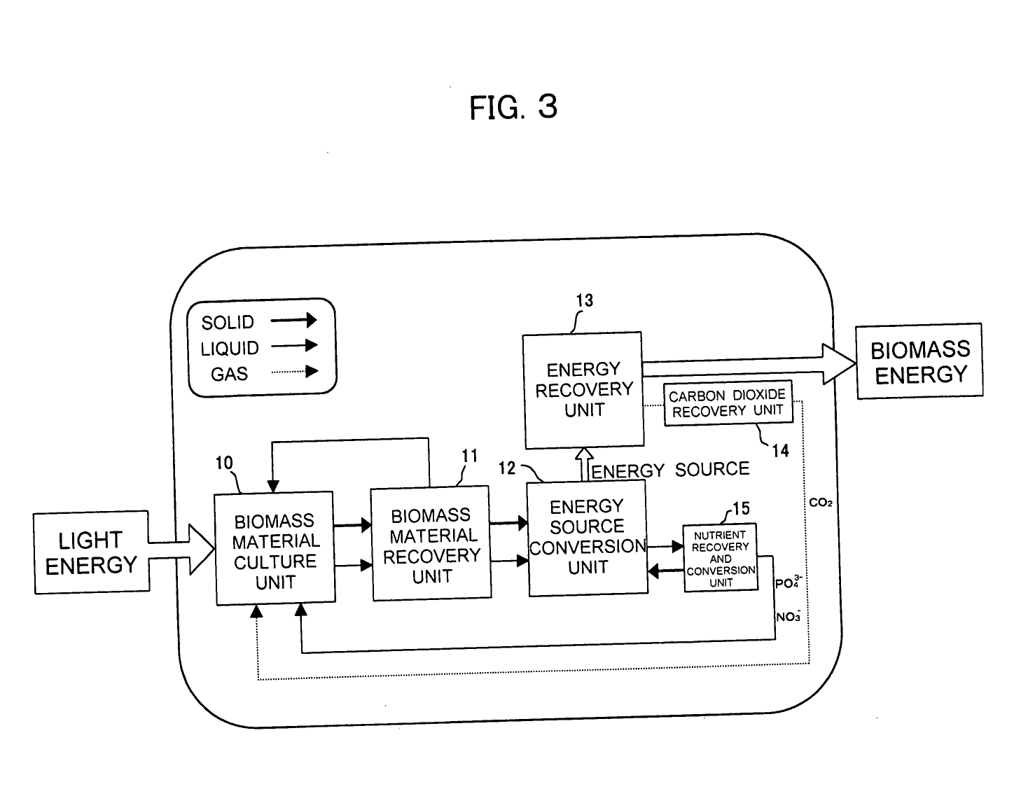

FIG. 3 is a view of the schematic constitution of a circulatory biomass energy recovery system according to the present embodiment.

The biomass energy recovery system according to the present embodiment has a culture unit 10, a biomass material recovery unit 11, an energy source conversion unit 12, an energy recovery unit 13, and a carbon dioxide recovery unit 14.

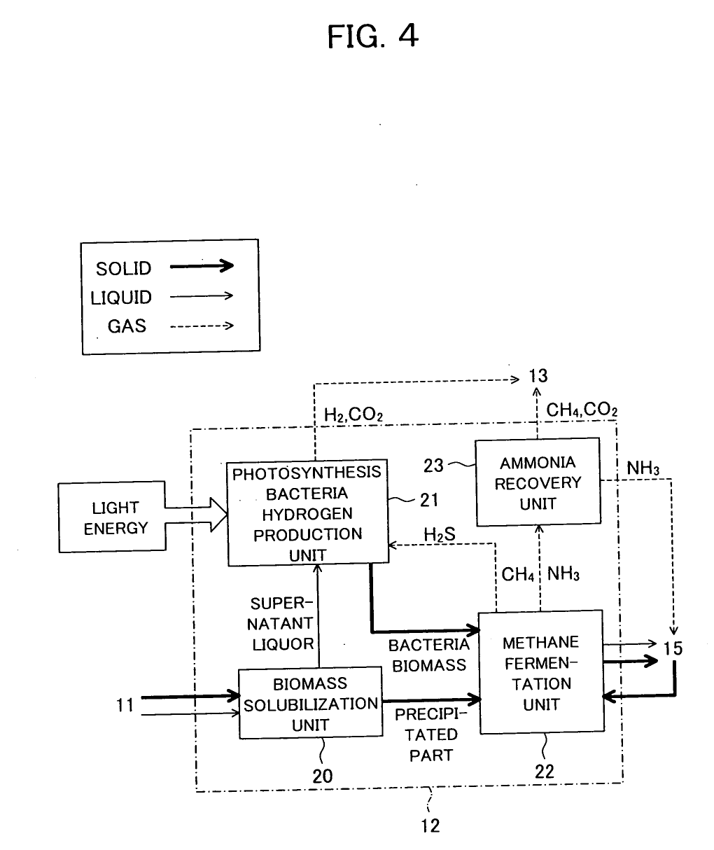

FIG. 4 is a view of the schematic constitution showing the constitution of an energy source conversion unit in FIG. 3 in the present embodiment in detail.

The culture unit 10 becomes the place for culturing the energy crop which becomes the biomass material, for example, a continuous culture unit for continuously culturing phytoplankton. When light energy such as sunlight is irradiated to the culture unit 10, the phytoplankton living in the water is cultured as the biomass material.

The phytoplankton described above is not particularly limited, but use can be made of, for example, Chlorella, Dunaliella, Chlamydomonas, Scenedesmus, Spirulina, and Porphyridium.

The culture unit 10 is configured as a water tank having an area of, for example, thousands to hundreds of thousands of m² and a depth of tens of cm to a few meters filled with the culture solution. An upper surface of the culture unit 10 is covered by transparent glass, acryl, or another lid material not passing UV-rays therethrough so as to shut the unit off from the open air. The gas atmosphere at the surface of the culture solution becomes a closed system and is controlled to an atmosphere having a chemical composition tailored to the growth of the phytoplankton as an energy crop.

In the culture unit 10, the culture solution is maintained at a salt concentration having a high nutrition. The growth rate of the phytoplankton is maintained at a high level.

Further, in order to raise the yield of the phytoplankton in the culture solution, the cell concentration is raised up to the physiologically and biologically limit value for the culturing.

Further, the culture solution is adjusted to a pH that enables the growing rate of the cells to become the maximum.

The biomass material recovery unit 11 recovers the biomass material cultured in the culture unit 10.

For example, it has is configured so that the phytoplankton is easily recovered by the transfer of the cultured phytoplankton to the biomass material recovery unit 11 together with the culture solution. It serves as a biomass material condensing and recovering unit condensing and recovering the biomass material. The phytoplankton controlled in growth rate to the maximum and proliferated in the culture unit is condensed and recovered.

In the above, the useful substances may be recovered in accordance with the type of the phytoplankton, and the obtained residue may be utilized as the biomass material as follows.

For example, various types of health foods can be produced from Chlorella, Spirulina, Dunaliella, Porphyridium, and so on. Even the residue after removing the useful substances is organic matter which becomes the biomass material.

The energy source conversion unit 12 converts the biomass material to an energy source capable of energy recovery.

The energy source conversion unit 12, as shown in FIG. 4, includes a hydrogen production unit 21 by photosynthesis bacteria using a biomass material and a methane fermentation unit 22 performing methane fermentation of the biomass material. Further, for example, the energy source conversion unit 12 includes a biomass solubilization unit 20 for solubilizing the biomass material and an ammonia recovery unit 23 for recovering the ammonia.

The biomass solubilization unit 20 solubilizes the produced phytoplankton or other biomass material to soluble organic matter such as organic acids which can be easily ingested by the photosynthesis bacteria to obtain a biomass solution.

It is configured so that the supernatant liquid of the biomass solution obtained by solubilizing the biomass is supplied to the hydrogen production unit 21 by the photosynthesis bacteria, and the precipitated part of the biomass solution is supplied to the methane fermentation unit 22.

The hydrogen production unit 21 by the photosynthesis bacteria uses the supernatant liquid of the biomass solution as the biomass material, irradiates it with light energy in the presence of photosynthesis bacteria, and converts it to hydrogen (H₂) forming an energy source by the action of the photosynthesis bacteria on the biomass material.

As the photosynthesis bacteria producing the hydrogen described above, there can be mentioned, for example, Rsp. molischianim, Rba. Sphaeroides, and Rps. Rubrum. Other than these, purple non-sulfur bacteria, purple sulfur bacteria and other purple bacteria, green sulfur bacteria, or other photosynthesis bacteria can be utilized as well.

By the photosynthesis bacteria described above, it is possible to use light as the energy source and use the biomass material as the substrate and completely decompose the organic matter to hydrogen and carbon dioxide by utilizing the light energy.

The hydrogen produced in the hydrogen production unit 21 by the photosynthesis bacteria is sent together with the carbon dioxide (CO₂) etc. to the energy recovery unit 13.

The methane fermentation unit 22 performs methane fermentation using the precipitated part of the biomass solution as the biomass material and converts it to methane (CH₄) which forms an energy source.

For example, the methane fermentation is carried out by decomposing the polysaccharides contained in the biomass, adding various methane bacteria known to generate methane as metabolites such as Methanococcus, Plethanosarcina, and Methanobacteriaceae, keeping the biomass at a predetermined temperature, and so on.

The ammonia recovery unit 23 recovers the gas component containing the methane gas and/or ammonia (NH₃) generated in the methane fermentation unit 22, separates the ammonia component and sends it to the nutrient recovery and conversion unit 15, and sends the methane gas component to the energy recovery unit 13.

In the above, preferably, for example, the photosynthesis bacteria obtained in the hydrogen production unit 21 by the photosynthesis bacteria and dead bodies thereof are supplied as the bacteria biomass and methane fermentation material to the methane fermentation unit 22.

The bacteria biomass contributes to the increase of the biomass material for the methane fermentation and enables the energy recovery efficiency to be raised.

Further, for example, the hydrogen sulfide (H₂S) obtained in the methane fermentation unit 22 is supplied to the hydrogen production unit 21 by the photosynthesis bacteria and utilized by the photosynthesis bacteria.

The harmful hydrogen sulfide generated in the methane fermentation unit 22 can be utilized in the hydrogen production unit 21 by the photosynthesis bacteria, therefore the load upon the environment due to its release outside of the system can be reduced.

The energy recovery unit 13 generates electric power using the effective energy source converted at the energy source conversion unit 12 or stores it as fuel itself and thereby recovers biomass energy.

For example, the energy recovery unit 13 includes an electric power generation unit generating electric power by burning the methane produced in the methane fermentation unit 22 and turning an electric power generation turbine.

Further, for example, the energy recovery unit 13 includes an electric power generation unit generating electric power by burning the hydrogen produced in the hydrogen production unit 21 by the photosynthesis bacteria.

When the electric power generation unit as described above is included, the biomass energy is recovered as electric power.

Further, preferably, a hydrogen recovery unit recovering the energy as the hydrogen produced in the hydrogen production unit by the photosynthesis bacteria is included as well.

In this case, the biomass energy is recovered in the state of the hydrogen as it is or in a form of a fuel cell etc.

The carbon dioxide recovery unit 14 returns the carbon dioxide produced in the energy recovery unit 13 and the energy source conversion unit 12 before it to the culture unit 10.

The carbon dioxide generated by the combustion etc. of the methane gas is recovered and sent to the culture unit 10 of the biomass material and subjected to the photosynthesis at the time of the phytoplankton culturing.

In this way, by returning the carbon dioxide recovered from the energy recovery unit 13 etc. to the culture unit 10, the energy recovery system becomes a closed type circulation system, so the composition of the gas can be freely controlled. For this reason, it becomes possible to raise the carbon dioxide partial pressure. In the circulatory biomass energy recovery system according to the present embodiment, the carbon fixing rate and the decomposition rate are rendered steady, so can be maintained.

In the circulatory biomass energy recovery system described above, the phytoplankton utilized as the biomass material contains, in addition to carbon (C), hydrogen (H), and oxygen (O), trace elements such as nitrogen (N) and phosphorus (P) as inorganic nutrient salts.

Three elements of the carbon (C), hydrogen (H), and oxygen (O) are recovered from the methane fermentation unit 22 of the energy source conversion unit 12 as the energy source methane gas, but the nitrogen (N), phosphorus (P), and other trace elements which form nutrient salts remain in the methane fermentation unit 22.

Therefore, the circulatory biomass energy recovery system according to the present embodiment further has a nutrient recovery and conversion unit 15 for recovering the nitrogen (N), phosphorus (P), and other trace elements remaining in the methane fermentation unit 22 as nutrients and converting the same to a form absorbed into the phytoplankton again according to need.

For example, the nutrient recovery and conversion unit 15 recovers the nitrogen (N), phosphorus (P), and other nutrients from the active sludge produced in the methane fermentation unit 22 and converts the same to phosphate ions, nitrate ions, or another form absorbed into the phytoplankton again. The obtained nutrients are returned to the culture unit 10 and utilized for the culturing of phytoplankton. The remaining excess sludge after recovering the nitrogen (N), phosphorus (P), and other nutrients is returned to the methane fermentation unit 22.

Further, the ammonia recovered at the ammonia recovery unit 23 is converted in the nutrient recovery and conversion unit 15 to a nutrient which can be utilized by the phytoplankton in the culture unit 10 and returned to the culture unit 10.

In this way, by circulating the nutrients recovered from the energy source conversion unit 12 to the culture unit 10, there is the advantage that it is no longer necessary to add a fertilizer.

According to the circulatory biomass energy recovery system of the present embodiment described above, the methane fermentation unit performing the methane fermentation of the biomass material and the hydrogen production unit by the photosynthesis bacteria using the biomass material are practically used. The photosynthesis bacteria generate the hydrogen, and, in addition, the photosynthesis bacteria themselves further becomes the biomass material of the methane fermentation and contribute to the increase of the recovery energy so enable the energy recovery efficiency to be raised.

A circulatory biomass energy recovery method using the circulatory biomass energy recovery system of the present embodiment described above will be explained next.

First, the culture unit filled with the culture solution cultures the phytoplankton as the biomass material.

Next, the biomass material is recovered from the culture unit.

Next, the biomass material is converted to an energy source enabling energy recovery.

Next, the energy is recovered from the energy source.

Further, the carbon dioxide generated in the energy recovery process is recovered and returned to the culture unit.

Here, the energy source conversion step includes the step of generating methane by methane fermentation of the biomass material and the step of producing hydrogen by the photosynthesis bacteria by using the biomass material.

Further, preferably, the energy source conversion step includes the step of solubilizing the biomass material, the supernatant liquid of the biomass solution obtained in the biomass solubilization is used for the hydrogen production by the photosynthesis bacteria, and the precipitated part of the biomass solution is used for the methane fermentation.

Further, preferably, the photosynthesis bacteria obtained in the hydrogen production step by the photosynthesis bacteria and dead bodies thereof are used as the methane fermentation material.

Further, at the time of the methane fermentation, preferably the obtained hydrogen sulfide is utilized by the photosynthesis bacteria in the hydrogen production step by the photosynthesis bacteria.

Further, at the time of energy recovery, the methane generated by the methane fermentation or the hydrogen produced in the hydrogen production by the photosynthesis bacteria is burned to generate electric power or the hydrogen produced in the hydrogen production by the photosynthesis bacteria is recovered so as to recover the biomass energy.

Further, according to the circulatory biomass energy recovery method according to the present embodiment, when converting the energy source, methane fermentation of the biomass material and the hydrogen production by the photosynthesis bacteria using the biomass material are carried out. The photosynthesis bacteria generate hydrogen, and, in addition, the photosynthesis bacteria themselves further become the biomass material of the methane fermentation and contribute to the increase of the recovery energy so enable the energy recovery efficiency to be raised for recovery of the biomass energy.

According to the circulatory biomass energy recovery system and method of the present embodiment described above, the following advantages can be given:

(1) The photosynthesis bacteria can simultaneously perform the carbon fixing and hydrogen production, therefore the energy can be recovered with a higher efficiency than the conventional plantation type biomass energy recovery system.

(2) Most of the organic matter obtained from the biomass of the phytoplankton can be recovered as energy in the form of hydrogen, so application to fuel cells etc. is also possible.

(3) The photosynthesis bacteria generate hydrogen while treating the organic acids and other organic matter, therefore can simultaneously perform wastewater treatment and energy recovery. This leads to a reduction of the size of wastewater treatment facilities in the system and enables reduction of the energy consumption in the system.

(4) The photosynthesis bacteria use the hydrogen sulfide as an electron donor, therefore the hydrogen sulfide discharged from the methane fermentation can be utilized in the photosynthesis bacteria culture tank.

### Second Embodiment

The present invention is a plantation type circulatory biomass energy recovery system and a circulatory biomass energy recovery method using same. An embodiment of the present invention will be explained below with reference to the drawings.

The circulatory biomass energy recovery system according to the present embodiment has the same constitution as that of the first embodiment shown in FIG. 3, that is, has the culture unit 10, biomass material recovery unit 11, energy source conversion unit 12, energy recovery unit 13, and carbon dioxide recovery unit 14.

The culture unit 10 becomes the place for culturing the energy crop which forms the biomass material, for example, a continuous culture unit for continuously culturing the phytoplankton. The phytoplankton living in the water is cultured as the biomass material in the culture unit 10.

In the present embodiment, a plurality of culture units are provided as the culture unit 10.

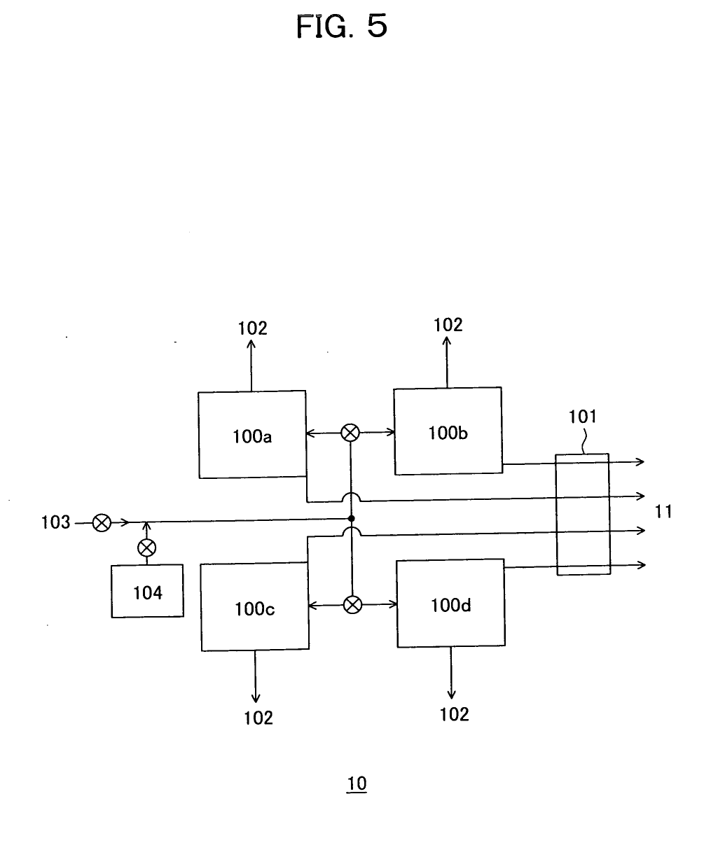

FIG. 5 is a view of the schematic constitution showing the constitution of the culture unit 10 according to the present embodiment in detail.

For example, a plurality of culture units 100a to 100d is provided. Each of these is connected to the biomass material recovery unit 11. The cultured phytoplankton can be recovered from each culture unit.

Each of the culture units 100a to 100d is configured as a water tank having an area of, for example, thousands to hundreds of thousands of m² and a depth of tens of cm to a few meters filled with the culture solution. An upper surface of the culture unit 10 is covered by transparent glass, acryl, or another lid material not passing UV-rays therethrough so as to shut the unit off from the open air. The gas atmosphere at the surface of the culture solution becomes a closed system and is controlled to an atmosphere having a chemical composition tailored to the growth of the phytoplankton as an energy crop.

In the culture unit 10, the culture solution is maintained at a salt concentration having a high nutrition. The growth rate of the phytoplankton is maintained at a high level.

Further, in order to raise the yield of the phytoplankton in the culture solution, the cell concentration is raised up to the physiologically and biologically limit value for the culturing.

Further, the culture solution is adjusted to a pH that enables the growing rate of the cells to become the maximum.

In the present embodiment, for example, preferably a monitor unit 101 monitoring the cultured state of the phytoplankton is provided so as to monitor each of the plurality of culture units 100a to 100d.

For example, the monitor unit 101 includes a measurement unit for measuring the fluorescence intensity of in vivo chlorophyll fluorescence by using the phytoplankton as a sample, and finding the fluorescence quantum yield.

For example, the spectrum of the in vivo chlorophyll fluorescence is positioned near 683 nm. In order to detect only the light near this wavelength, a spectral filter is used. However, when the in vivo chlorophyll fluorescence is measured in a state where light in the visible zone (background light) effective for the photosynthesis is irradiated to the phytoplankton, the detector detects the background light and the chlorophyll fluorescence together, therefore the quantum yield of the fluorescence cannot be found. For example, in a PAM type fluorescence photometer, a light emitting diode is repeatedly turned on/off in an extremely short time for pulse modulation at the frequencies of the measurement light, that is, excitation light, and the background light.

In order to raise an S/N ratio of the chlorophyll fluorescence detected, the measurement light is desirably given a high energy and a high frequency. However, in order to measure the chlorophyll fluorescence in a state where the P680 is as open as possible, the cumulative energy of the measurement light is lowered by using light having a low frequency.

The measurement light has, for example, a center wavelength of 650 nm, a flash interval of 5 microseconds, and 8 to 688 Hz (for example, 18 Hz).

As described above, preferably, the cultured state of the phytoplankton is monitored by, for example, a PAM type fluorescence photometer. When the cultured state of the phytoplankton becomes lower than the target level, the culture solution is replaced by a new one in any culture unit where the cultured state becomes lower than the target level among the plurality of culture units.

For example, as shown in FIG. 5, each of the culture units 100a to 100d is independently connected to the drainage system 102, and the culture units 100a to 100d are connected to each other.

In the above, only the culture solution in a culture tank in which sudden death occurs is discarded. Parts of the culture solutions containing phytoplankton are transported from the other culture units to that culture unit to enable continuous culturing. The culture solution can be transported among other culture units as well.

Alternatively, as shown in FIG. 5, each of the culture units 100a to 100d is independently connected to the drainage system 102, a new water supply source 103 is connected to an upstream side, and a wild strain culture tank 104 of the phytoplankton is connected between that new water supply source 103 and each of the culture units 100a to 100d.

For example, only the culture solution in a culture tank where sudden death occurred may be discarded and new water and a wild strain may be supplied to replace the culture solution by a new one.

Further, for example, the wild strain culture tank 104 may periodically supply a strain of fresh phytoplankton to each of the culture units 100a to 100d.

In the present embodiment, the plurality of culture units may be independently provided or the culture units can be realized by partitioning a single culture tank into a plurality of regions by partitioning members.

In the present embodiment, preferably the culture unit is a continuous culture unit continuously culturing the phytoplankton.

When continuous culturing, problems such as sudden death of the phytoplankton occur, but the stable supply of the phytoplankton can be realized in the present embodiment even when sudden death occurs.

The biomass material recovery unit 11 recovers the biomass material cultured in the culture unit 10.

For example, it is configured so that the phytoplankton is easily recovered by the transfer of the cultured phytoplankton together with the culture solution to the biomass material recovery unit 11 and becomes a biomass material condensing and recovering unit condensing and recovering the biomass material. The phytoplankton controlled in growth rate to the maximum and proliferated in the culture unit is condensed and recovered.

In the above, useful substances may be recovered in accordance with the type of the phytoplankton, and the obtained residue may be utilized as the biomass material as follows.

For example, various types of health foods can be produced from Chlorella, Spirulina, Dunaliella, Porphyridium, and so on. Even the residue after removing the useful substances is organic matter which becomes the biomass material.

The energy source conversion unit 12 converts the biomass material to an energy source capable of energy recovery.

The energy source conversion unit 12 becomes, for example, the methane fermentation unit performing methane fermentation of the biomass material. The methane gas is obtained from the phytoplankton material of the biomass energy by the methane fermentation. For example, the methane fermentation is carried out by decomposing the polysaccharides contained in the biomass, adding various methane bacteria known to generate methane as metabolites such as Methanococcus, Methanosarcina, and Methanobacteriaceae, keeping the biomass at a predetermined temperature, and so on.

Alternatively, it may be made an alcohol conversion unit etc. converting this to ethanol, methanol, or the like may. For example, alcohol is fermented by decomposing the polysaccharides contained in the biomass, adding yeast belonging to Saccaromyces etc., keeping the biomass at a predetermined temperature, etc.

The energy recovery unit 13 generates electric power using the effective energy source converted at the energy source conversion unit 12 or stores it as fuel itself and thereby recovers biomass energy.

For example, when the energy source conversion unit 12 is a methane fermentation unit, the energy recovery unit 13 may be made an electric power generation unit generating electric power by burning the methane and turning an electric power generation turbine.

The carbon dioxide recovery unit 14 returns the carbon dioxide produced in the energy recovery unit to the culture unit 10.

The carbon dioxide generated in the energy recovery unit 13 by burning the methane gas etc. is recovered and sent to the culture unit 10 of the biomass material and subjected to photosynthesis at the time of the culturing of the phytoplankton.

In the circulatory biomass energy recovery system described above, for example, the methane gas, ethanol, methanol, or the like generated as the energy source as described above is composed of carbon (C), hydrogen (H), and oxygen (O).

The phytoplankton utilized as the material not only contains carbon (C), hydrogen (H), and oxygen (O), but also further contains nitrogen (N), phosphorus (P), and other trace elements entering as inorganic nutrient salts.

Three elements of carbon (C), hydrogen (H), and oxygen (O) are recovered from the liquid phase in the energy source conversion unit 12 as energy and thereby removed, but the nitrogen (N), phosphorus (P), and other trace elements which form nutrient salts remaining in the liquid phase in the energy source conversion unit.

Therefore, the circulatory biomass energy recovery system according to the present embodiment further has a nutrient recovery and conversion unit 15 for further recovering the nitrogen (N), phosphorus (P), and other trace elements remaining in the liquid phase in the energy source conversion unit 12 as nutrients and converting the same to such form that is absorbed into the phytoplankton again according to need.

For example, the nutrients are recovered as salts containing nitrate ions, phosphate ions, etc., returned to the culture unit 10, and utilized for culturing the phytoplankton.

Further, even in a case where ammonia is generated in the energy source conversion unit 12, the ammonia is recovered and is converted in the nutrient recovery and conversion unit 15 to nutrients that can be utilized by the phytoplankton in the culture unit 10 and when are then returned to the culture unit 10.

In the circulatory biomass energy recovery system described above, when the light energy of sunlight is irradiated to the culture unit 10, the phytoplankton forming the biomass material is cultured in the culture solution of the culture unit 10.

The cultured phytoplankton is transferred together with the culture solution to the biomass material recovery unit 11 where the phytoplankton is recovered and the wet biomass material of the phytoplankton is obtained. Excess culture solution is returned to the culture unit 10.

The wet biomass material is charged into the energy source conversion unit 12 and converted to methane gas, alcohol, or another energy source.

From the obtained energy source, the energy recovery unit 13 recovers energy as biomass energy.

Here, the carbon dioxide gas generated in the energy recovery unit 13 due to the combustion of the methane gas etc. is recovered by the carbon dioxide recovery unit 14 and sent to the culture unit 10 so as to be subjected to the photosynthesis of the phytoplankton.

In this way, by returning the carbon dioxide recovered from the energy recovery unit 13 to the culture unit 10, the energy recovery system becomes a closed type circulation system, so the composition of the gas can be freely controlled. For this reason, it becomes possible to raise the carbon dioxide partial pressure. In the circulatory biomass energy recovery system according to the present embodiment, the carbon fixing rate and the decomposition rate are rendered steady, so can be maintained.

Further, the nutrients containing the nitrogen (N), phosphorus (P), etc. generated in the energy source conversion unit 12 are converted to an aqueous solution of nutrient salts containing nitrate ions, phosphate ions, etc. of form reabsorbed into the phytoplankton in the nutrient recovery and conversion unit 15 and returned to the culture unit 10 for utilization for culturing the phytoplankton. The excess solid component after recovering the nutrients is returned from the nutrient recovery and conversion unit 15 to the energy source conversion unit 12.

In this way, by circulating the nutrients recovered from the energy source conversion unit 12 to the culture unit 10, there is the advantage that the necessity of adding a fertilizer is eliminated.

According to the circulatory biomass energy recovery system of the present embodiment described above, by having a plurality of culture units, even when sudden death of the phytoplankton occurs in any culture unit among those, the influence exerted upon the other culture units can be avoided, stable supply of the phytoplankton as the biomass material becomes possible, and stabilization of the electric power or other energy supply can be realized.

A circulatory biomass energy recovery method using the circulatory biomass energy recovery system of the present embodiment described above will be explained next.

First, phytoplankton is cultured as the biomass material in a plurality of culture units filled with the culture solution.

Next, the biomass material cultured in the plurality of culture units is recovered.

Next, the biomass material is converted to an energy source capable of energy recovery.

Next, the energy is recovered from the energy source.

After this, the carbon dioxide generated in the energy recovery step is recovered and returned to the culture units.

In the circulatory biomass energy recovery method of the present embodiment, the step of culturing the phytoplankton monitors the culture stated of the phytoplankton in each of the plurality of culture units.

For example, in the step of culturing the phytoplankton, the fluorescence intensity of the in vivo chlorophyll fluorescence is measured by using the phytoplankton as a sample, and the fluorescence quantum yield is found.

Here, when the cultured state of the phytoplankton becomes lower than the target level, the culture solution is replaced by a new one in any culture unit where the culture stated becomes lower than the target level among the plurality of culture units, so continuous culturing is carried out.

Further, as the plurality of culture units, it is possible to use a plurality of culture units constituted by partitioning one culture tank into a plurality of regions by partitioning members.

Further, in the step of culturing the phytoplankton, preferably the phytoplankton is continuously cultured in the culture units.

Further, according to the circulatory biomass energy recovery method according to the present embodiment, by culturing the phytoplankton in the plurality of culture units, even when sudden death of the phytoplankton occurs in any culture unit among those, the influence exerted upon the other culture units can be avoided, stable supply of the phytoplankton as the biomass material becomes possible, and stabilization of the electric power or other energy supply can be realized.

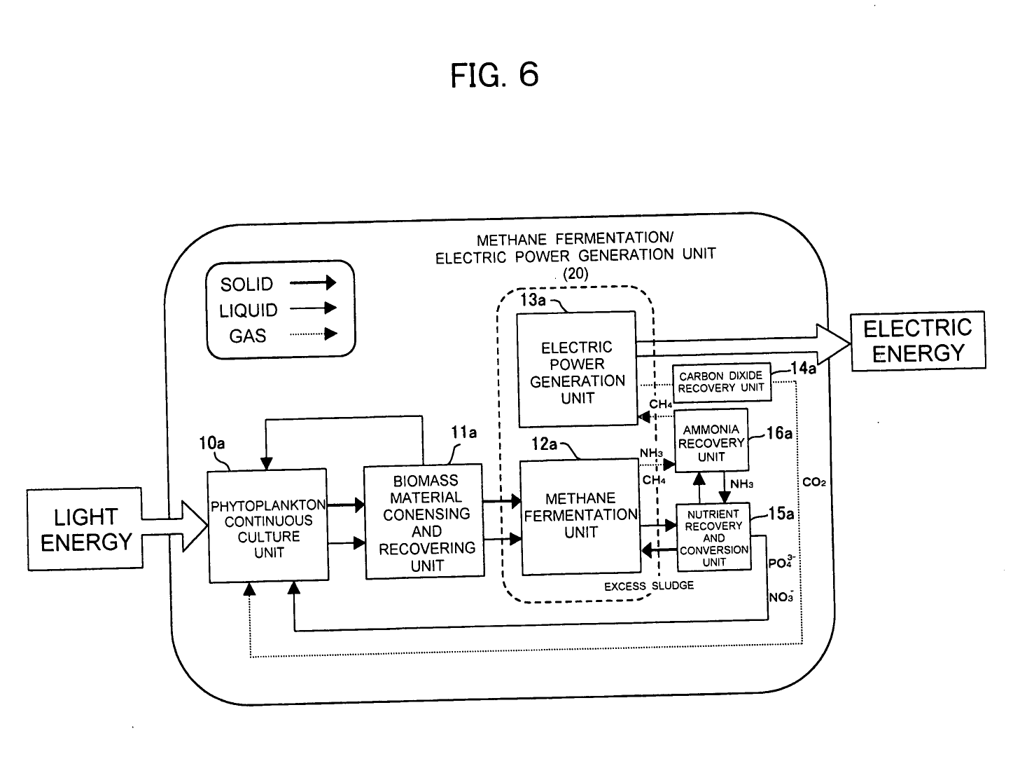

FIG. 6 is a view of the schematic constitution of a circulatory biomass energy recovery system given a more concrete constitution in the present embodiment.

The circulatory biomass energy recovery system described above has a continuous culture unit 10a of the phytoplankton, a biomass material condensing and recovering unit 11a, a methane fermentation unit 12a, an electric power generation unit 13a, a carbon dioxide recovery unit 14a, a nutrient recovery and conversion unit 15a, and an ammonia recovery unit 16a.

The continuous culture unit 10a of the phytoplankton is configured as shown in FIG. 5 described above and continuously cultures the phytoplankton forming the biomass material.

The phytoplankton is not particularly limited. Use can be made of, for example, Chlorella, Dunaliella, Chlamydomonas, Scenedesmus, and Spirulina.

The biomass material condensing and recovering unit 11a condenses and recovers the biomass material cultured in the continuous culture unit 10a of the phytoplankton.

The methane fermentation unit 12a performs methane fermentation of the biomass material and converts it to the energy source methane.

The ammonia recovery unit 16a recovers the gas component containing the methane gas and ammonia generated in the methane fermentation unit 12a, separates the ammonia component and sends it to the nutrient recovery and conversion unit 15a, and sends the methane gas component to the electric power generation unit 13a.

The electric power generation unit 13a burns the energy source methane gas to turn the electric power generation turbine and generate electric power and thereby recovers electrical energy.

Note that it is possible to assemble a system in which the methane fermentation unit 12a and the electric power generation unit 13a become integral as a methane fermentation and electric power generation unit 30.

Further, the carbon dioxide generated by the combustion of the methane gas at the electric power generation unit 13a is recovered by the carbon dioxide recovery unit 14a and sent to the continuous culture unit 10a of the phytoplankton where it is subjected to the photosynthesis of the phytoplankton.

Further, the nutrient recovery and conversion unit 15a recovers the nitrogen (N), phosphorus (P), and other nutrients from the active sludge produced in the methane fermentation unit 12a, converts the same to phosphate ions, nitrate ions, or another form that is absorbed by the phytoplankton, and returns the obtained nutrients to the continuous culture unit 10a of the phytoplankton. The remaining excess sludge after recovering the nutrients such as nitrogen (N) and phosphorus (P) is returned to the methane fermentation unit 12a.

The ammonia component recovered from the ammonia recovery unit 16a is converted to a form that is absorbed into the phytoplankton in the same way and returned to the continuous culture unit 10a of the phytoplankton.

According to the circulatory biomass energy recovery system of the present embodiment described above, the following advantages can be given:

(1) The biomass material of the phytoplankton can be stably produced and electric power and other energy can be stably supplied.

(2) Several types of seaweed can be simultaneously produced, therefore a plurality of effective substances produced by the phytoplankton can be simultaneously recovered.

The present invention is not limited to the above embodiments.

For example, as the energy recovery, other than electric power generation using the energy source, the energy can be stored as the fuel itself.

Other than that, various modifications are possible within a range not out of the gist of the present invention.

### INDUSTRIAL APPLICABILITY

The biomass energy recovery system of the present invention can be applied as an environmentally friendly system for recovering energy sources not discharging carbon dioxide and other global warming gases.

The biomass energy recovery method of the present invention can be applied as an environmentally friendly method for recovering energy.

1. A circulatory biomass energy recovery system comprising:
a culture unit filled with a culture solution for culturing phytoplankton as a biomass material,
a biomass material recovery unit for recovering the biomass material from the culture unit,
an energy source conversion unit for converting the biomass material to an energy source capable of energy recovery,
an energy recovery unit for recovering energy from the energy source converted at the energy source conversion unit, and
a carbon dioxide recovery unit for returning the carbon dioxide produced in the energy recovery unit to the culture unit, wherein
the energy source conversion unit includes a methane fermentation unit for performing methane fermentation of the biomass material and a hydrogen production unit by photosynthesis bacteria using the biomass material.

2. A circulatory biomass energy recovery system as set forth in claim 1, wherein
the energy source conversion unit includes a biomass solubilization unit for solubilizing the biomass material,
a supernatant liquid of the biomass solution obtained by the biomass solubilization is supplied to the hydrogen production unit by the photosynthesis bacteria, and
a precipitated part of the biomass solution is supplied to the methane fermentation unit.

3. A circulatory biomass energy recovery system as set forth in claim 1, wherein
the photosynthesis bacteria obtained in the hydrogen production unit by the photosynthesis bacteria and dead bodies thereof are supplied as the methane fermentation material to the methane fermentation unit.

4. A circulatory biomass energy recovery system as set forth in claim 1, wherein hydrogen sulfide obtained in the methane fermentation unit is supplied to the hydrogen production unit by the photosynthesis bacteria and is utilized by the photosynthesis bacteria.

5. A circulatory biomass energy recovery system as set forth in claim 1, wherein the energy recovery unit includes an electric power generation unit for generating electric power by burning the methane generated in the methane fermentation unit.

6. A circulatory biomass energy recovery system as set forth in claim 1, wherein the energy recovery unit includes an electric power generation unit for generating electric power by burning the hydrogen produced in the hydrogen production unit by the photosynthesis bacteria.

7. A circulatory biomass energy recovery system as set forth in claim 1, wherein the energy recovery unit includes a hydrogen recovery unit for recovering the hydrogen produced in the hydrogen production unit by the photosynthesis bacteria.

8. A circulatory biomass energy recovery method comprising:
a step of culturing phytoplankton as a biomass material in a culture unit filled with a culture solution,
a step of recovering the biomass material from the culture unit,
an energy source converting step of converting the biomass material to an energy source capable of energy recovery,
an energy recovery step of recovering the energy from the energy source, and
a step of recovering and returning the carbon dioxide generated in the energy recovery step to the culture unit, wherein
the energy source converting step includes a step of generating methane by methane fermentation of the biomass material and a step of producing hydrogen by photosynthesis bacteria using the biomass material.

9. A circulatory biomass energy recovery method as set forth in claim 8, wherein
the energy source converting step includes a step of solubilizing the biomass material,
a supernatant liquid of a biomass solution obtained by the solubilization of the biomass is used for hydrogen production by the photosynthesis bacteria, and
a precipitated part of the biomass solution is used for the methane fermentation.

10. A circulatory biomass energy recovery method as set forth in claim 8, wherein the photosynthesis bacteria obtained in the hydrogen production step by the photosynthesis bacteria and dead bodied thereof are used as the methane fermentation material.

11. A circulatory biomass energy recovery method as set forth in claim 8, wherein the hydrogen sulfide obtained in the methane fermentation step is utilized by the photosynthesis bacteria in the hydrogen production step by the photosynthesis bacteria.

12. A circulatory biomass energy recovery method as set forth in claim 8, wherein the energy recovery step includes a step of generating electric power by burning the methane produced by the methane fermentation.

13. A circulatory biomass energy recovery method as set forth in claim 8, wherein the energy recovery step includes a step of generating electric power by burning the hydrogen produced in the hydrogen production by the photosynthesis bacteria.

14. A circulatory biomass energy recovery method as set forth in claim 8, wherein the energy recovery step includes a step of recovering the hydrogen produced in the hydrogen production by the photosynthesis bacteria.

15. A circulatory biomass energy recovery system comprising:
a plurality of culture units filled with a culture solution for culturing phytoplankton as a biomass material,
a biomass material recovery unit for recovering the biomass material cultured in the plurality of culture units,
an energy source conversion unit for converting the biomass material to an energy source capable of energy recovery,
an energy recovery unit for recovering the energy from the energy source converted at the energy source conversion unit, and
a carbon dioxide recovery unit for returning the carbon dioxide produced in the energy recovery unit to the culture units.

16. A circulatory biomass energy recovery system as set forth in claim 15, wherein the plurality of culture units are constituted by partitioning one culture tank into a plurality of regions by a partition member.

17. A circulatory biomass energy recovery system as set forth in claim 15, wherein a monitor unit for monitoring a cultured state of the phytoplankton in each of the plurality of culture units is provided.

18. A circulatory biomass energy recovery system as set forth in claim 17, wherein the monitor unit includes a measurement unit for measuring a fluorescence intensity of an in vivo chlorophyll fluorescence by using the phytoplankton as a sample and finding a fluorescence quantum yield.

19. A circulatory biomass energy recovery system as set forth in claim 17, wherein, when the cultured state of the phytoplankton becomes lower than a target level, the culture solution is replaced by a new one in any culture unit where the cultured state becomes lower than the target level among the plurality of culture units.

20. A circulatory biomass energy recovery system as set forth in claim 15, wherein the culture units are continuous culture units for continuously culturing the phytoplankton.

21. A circulatory biomass energy recovery method comprising:
a step of culturing phytoplankton as a biomass material in a plurality of culture units filled with a culture solution,
a step of recovering the biomass material cultured in the culture units,
an energy source converting step of converting the biomass material to an energy source capable of energy recovery,
an energy recovery step of recovering the energy from the energy source, and
a step of recovering and returning carbon dioxide generated in the energy recovery step to the culture units.

22. A circulatory biomass energy recovery method as set forth in claim 21, wherein as said plurality of culture units, a plurality of culture units constituted by partitioning of one culture tank into a plurality of regions by a partitioning member is used.

23. A circulatory biomass energy recovery method as set forth in claim 21, wherein the step of culturing the phytoplankton is carried out while monitoring the cultured state of the phytoplankton in each of the plurality of culture units.

24. A circulatory biomass energy recovery method as set forth in claim 23, wherein in the step of culturing the phytoplankton a fluorescence intensity of in vivo chlorophyll fluorescence is measured by using the phytoplankton as a sample and a fluorescence quantum yield is found.

25. A circulatory biomass energy recovery method as set forth in claim 23, wherein in the step of culturing the phytoplankton, when the cultured state of the phytoplankton becomes lower than a target level, the culture solution is replaced by a new one in any culture unit where the cultured state becomes lower than the target level among the plurality of culture units.

26. A circulatory biomass energy recovery method as set forth in claim 21, wherein in the step of culturing the phytoplankton the phytoplankton is continuously cultured in the culture units.
